(19) United States
(12) Patent Application Publication
(10) Pub. No.: US 2007/0255345 A1
(43) Pub. Date: Nov. 1, 2007

(54) Method and System for Triggering an Implantable Medical Device for Risk Stratification Measurements

(76) Inventor: Paul G. Krause, St. Louis Park, MN (US)

Correspondence Address: MEDTRONIC, INC., 710 MEDTRONIC PARKWAY NE, MINNEAPOLIS, MN, 55432-9924, US

(21) Appl. No.: 11/380,307

(22) Filed: Apr. 26, 2006

(51) Int. Cl. A61N 1/00

(52) U.S. Cl. 607/59

(57) ABSTRACT

A method and system for triggering an implantable medical device for risk stratification measurements is disclosed. An implantable medical device having a hermetically sealed enclosure and memory disposed within the hermetically sealed enclosure. The device is programmed to record a physiological signal in response to at least one of a plurality of risk stratification measurement triggers. The stored signal is useful for implementing a variety of risk stratification for sudden cardiac death techniques.

### FIELD OF THE INVENTION

The present invention relates to implantable medical devices (IMDs).

### BACKGROUND

Risk stratification is an important tool to help determine which patients are most at risk for sudden cardiac death. Identification of such patients allows the health care system to focus on the patients most at risk. Risk stratification techniques include T-wave alternans, ischemia detection via ST segment analysis, ischemia detection via high-frequency analysis, signal-averaged QRS complex, QT dynamicity, QT dispersion, QT and/or T-wave morphology, and heart rate turbulence.

The data generally required to utilize these techniques is currently obtained via external electrocardiogram (ECG) electrodes. For example, patients are monitored through external devices such as Holter monitors or event recorders which record ECGs though electrodes attached to the skin. Such devices can make recordings over periods of time from days to a week or more. However, they are bulky and must be toted around by the patient, thus interfering with the patient's normal life and making them impractical for long term use. In addition, they may limit physical activities and must be removed during activities such as showering. Patients may also complain of skin irritation. Because the monitors must be worn for extended periods of time, these patient annoyances may result in poor patient compliance, decreasing their usefulness.

### BRIEF DESCRIPTION OF THE DRAWINGS

FIG. 1 shows a simplified front plan schematic view of an IMD in accordance with an embodiment of the invention;

FIG. 2 shows a simplified schematic of various components of an IMD in accordance with an embodiment of the invention; and

FIG. 3 shows a schematic flow diagram in accordance with an embodiment of the invention.

### DETAILED DESCRIPTION OF THE ILLUSTRATED EMBODIMENTS

The following discussion is presented to enable a person skilled in the art to make and use the invention. Various modifications to the illustrated embodiments will be readily apparent to those skilled in the art, and the generic principles herein may be applied to other embodiments and applications without departing from the spirit and scope of the invention as defined by the appended claims. Thus, the invention is not intended to be limited to the embodiments shown, but is to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the invention. Skilled artisans will recognize the examples provided herein have many useful alternatives that fall within the scope of the invention.

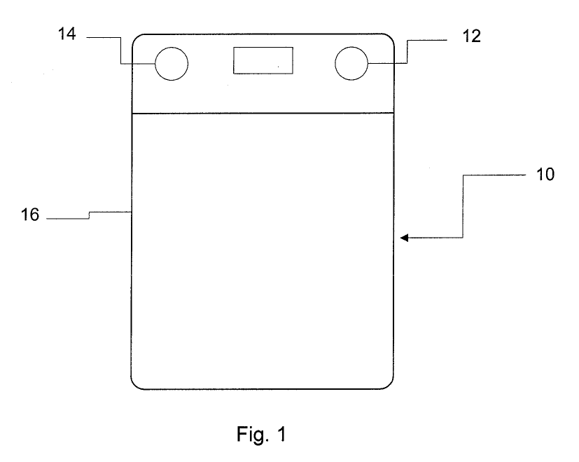

FIG. 1 is a simplified schematic view of an embodiment of an implantable medical device (“IMD”) 10. IMD 10 shown in FIG. 1 is an implantable loop recorder comprising a pair of sensing electrodes 12, 14 on a hermetically sealed enclosure 16. Such an IMD is capable of implantation within a mammalian body. For example, the IMD can be implanted subdermally such that the electrodes are in non-touching proximity to a mammalian heart. The sensing electrodes sense electrical signals attendant to the depolarization and re-polarization of the heart.

In one embodiment, the IMD provides long term monitoring of a physiological signal, such as an electrocardiogram (ECG) (i.e., monitoring of the subcutaneous (or intramuscular or submuscular) ECG) or electrogram (EGM). The device may continuously record and monitor the subcutaneous ECG in an endless loop of memory. The device may be triggered to save/retain a certain number of minutes of ECG recording. The device may itself trigger this recording after interpreting the signal it is receiving. This is referred to as autotriggering. In many instances, the IMD is programmed to retain signals associated with an event, such as an arrhythmia. In some embodiments, the IMD is programmed to save a signal in response to at least one of a plurality of risk stratification measurement triggers, as discussed further below. In such embodiments, the IMD will store information useful for implementing a variety of risk stratification techniques.

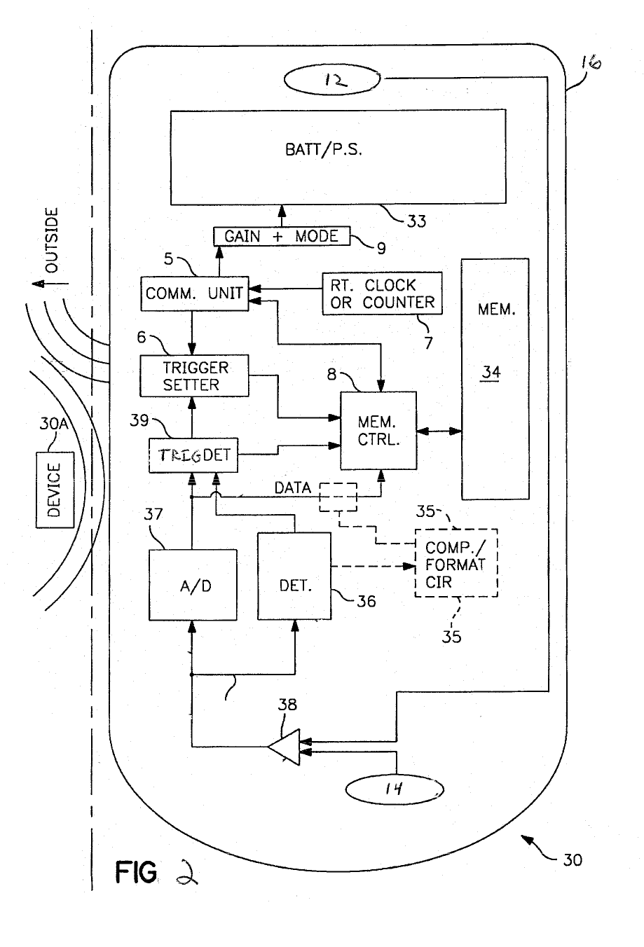

In FIG. 2, a circuit model 30 is illustrated in an outline of an implantable device enclosure 16. In this embodiment, electrodes 12 and 14 bring signal from the body to an input mechanism 38, here drawn as a differential amplifier for simplicity only, the output of which is fed to a detector 36 and an A/D converter 37. Both these circuits 36 and 37 supply output to a triggering determination circuit 39, which in this preferred embodiment supplies the autotrigger signal to the trigger setting circuit 6. The data output from the analog to digital converter may be converted, compressed, formatted and marked or reformulated if desired in a circuit 35 before the data is ready for input into the memory 34. The memory control circuit 8 receives input from the A/D converter, with or without conversion from circuit 35, from the auto triggering determination circuit 39 as well as signals from the trigger setter circuit 6. The trigger setter circuit may also be controlled by a communications unit 5 which operates to receive and decode signals from the outside of the implant 30 that are telemetered or otherwise communicated in by a user. This communications unit 5 will also be able to communicate with the memory controller to request the offloading of memory data for analysis by an outside device. It should contain an antenna or other transceiver device or circuitry to communicate with an outside device such as device 30A. A clock or counter circuit 7 reports the time since start or real time to the outside interrogator device 30A contemporaneously with a data offloading session so that the events recorded in memory 34 may be temporally pinpointed.

Alternatives to this overall design may be considered, for example by using a microprocessor to accomplish some or all of the functions of circuits 6, 8, 39, and 35. For a more detailed description of the components shown in FIG. 2, refer to U.S. Pat. No. 5,987,352, the relevant parts of which are hereby incorporated by reference.

Further, although IMD 10 is described as a implantable loop recorder, those of ordinary skill in the art will appreciate that the invention may be advantageously practiced in connection with numerous other types of IMDs, such as pacemakers, implantable cardioverter defibrillators (ICDs), PCD pacemakers/cardioverters/defibrillators, oxygen sensing devices, nerve stimulators, muscle stimulators, drug pumps, implantable monitoring devices, or combinations thereof. In addition, although the sensor is primarily referred to as an electrode, any sensor could be used with the IMD, such as a pressure sensor. Further, although the physiological signal is primarily referred to as an ECG, is should be understood that other physiological signals are included within the scope of the invention, such as electrograms.

Embodiments of the invention include an IMD with the ability to identify the presence of at least one of a plurality of risk stratification measurement triggers and trigger physiological signal (e.g., ECG) storage at a rate and rhythm that is suitable for sudden cardiac death (SCD) risk stratification measurements. Many risk stratification methods exist; however, many are too computationally complex to be practically implemented directly in an implanted device. An alternative means of implementation is to use the IMD to store an ECG signal that is suitable for processing and allow an external software platform (such as that on a Medtronic 2090 programmer, Medtronic CareLink application, or some other data transfer or analysis system) to calculate the risk stratification metric.

In some embodiments, the invention includes a system and method for identifying the presence of at least one of a plurality of risk stratification measurement triggers and triggering physiological signal (e.g., ECG) storage in an implanted medical device that provides data sufficient for calculation of several of the most common SCD risk stratification techniques. In some embodiments, the method includes the steps of sensing a physiological signal, identifying the presence of at least one of a plurality of risk stratification measurement triggers, storing the physiological signal in response to a trigger, prioritization of which signals to preserve if memory is limited, transfer of the physiological signal for processing, and/or translation of the signal to a common format for third-party software analysis.

There are many known methods to stratify SCD risk. For a risk stratification-focused trigger to be feasible in an implanted product, it is impractical as well as unnecessary to provide unique triggers for each possible method. Rather, a few triggers that are capable of storing signal that is suitable for the most common/useful techniques can be provided. Table 1 provides a list of the common techniques, with representative requirements given for the ECG signal that is used to compute each. It should be noted that these examples are not the only, or necessarily the optimal, methods of risk stratification. Rather, they are merely representative of risk stratification methods known in the art.

TABLE 1
Risk Stratification Summary with ECG Requirements

| | Method of measurement/ | |
|---|---|---|
| Technique | computation | ECG signal requirement |
| T-wave | ECG signal is collected | ECG recorded during |
| alternans | over increasing rates. | normal sinus rhythm at |
| | (Rates are increased | rest and during elevated |
| | using exercise or | rates. ECG is usually |
| | atrial pacing.) | recorded at a variety of |
| | Beat-to-beat alternating | elevated rates |
| | variation in T-wave | Can be performed with |
| | morphology is evaluated | as little as seven beats, |
| | using either a | though typically |
| | frequency-domain or | approximately 128 |
| | time-domain technique. | beats are required. |
| | Alternans will be | Bandwidth of at least |
| | evident in almost every | 0.6 to 50 Hz |
| | patient at a high rate. | Sampling rate of at |
| | However, if alternans are | least 250 Hz to ensure |
| | present only at moderate | adequate alignment |
| | rates, the test is | of QRS complexes across |
| | considered to be a | several beats |
| | positive finding and | Linear phase response |
| | risk of SCD may be higher. | of ECG signal from |
| | | 0.3 to 50 Hz, which |
| | | typically is |
| | | accomplished by |
| | | ensuring a bandwidth |
| | | extending down to 0.05 Hz. |
| | | Amplitude sampling |
| | | resolution ≦1.2 uV. |
| Ischemia | ECG signal is recorded | ECG recorded during |
| detection | during either | normal sinus rhythm at |
| via ST | ambulatory 24-hour | rest and/or during |
| segment | recordings or during | elevated rates. Enough |
| analysis | exercise. | beats need to be |
| | The degree to which | recorded to provide a |
| | the ST segment is | comparison between |
| | elevated or depressed | nominal ST segments and |
| | and the segment's | elevated/depressed ST |
| | morphology are used as | segments Minimal |
| | indications of | distortion of an |
| | ischemia. A finding of | ischemic QRST test |
| | ischemia greatly | signal, which typically |
| | increases SCD risk. | is accomplished by |
| | | ensuring a bandwidth |
| | | extending down to 0.05 Hz |
| | | Amplitude sampling |
| | | resolution <25 uV |
| | | Ideally, multiple vectors |
| | | would be recorded since |
| | | ST segment changes |
| | | during ischemia are |
| | | not always seen by |
| | | all ECG vectors. |
| | | However, this is not |
| | | required. |
| Ischemia | ECG signal is recorded | ECG recorded during |
| detection | during either | normal sinus rhythm |
| via | ambulatory 24-hour | at rest and/or during |
| high- | recordings or during | elevated rates for |
| frequency | exercise. | approximately 200 beats. |
| analysis | The signal is averaged | Sampling rate of at least |
| | across several beats, | 500 Hz, with an ideal |
| | filtered between | sampling rate of 1000 Hz. |
| | 150-250 Hz, and the | Amplitude resolution |
| | remaining signal's | on the order of 1 uV |
| | morphology is used as | Ideally, multiple vectors |
| | an indication of | would be recorded since |
| | ischemia. A finding of | ST segment changes |
| | ischemia greatly | during ischemia are |
| | increases SCD risk. | not always seen by |
| | | all ECG vectors. |
| | | However, this is not |
| | | required. |
| Signal- | QRS complexes are | Recording of normal |
| averaged | collected across | sinus rhythm for |
| QRS | several beats, aligned, | approximately 200 |
| complex | and averaged. Used | to 600 beats |
| | as an indirect | Noise <=1 uV |
| | measurement of late | Sampling rate of at |
| | potentials, which | least 200 Hz to avoid |
| | can be a predictor | aliasing QRS complex |
| | of SCD risk. | |
| QT | QT intervals from | ECG recorded during |
| Dynamicity | ECG are measured using | normal sinus rhythm |
| | consistent fiducial | for at least several |
| | points on the Q-wave | hours - possibly as |
| | and T-wave. RR | much as an entire 24 |
| | intervals are also | hour period. |
| | measured. The QT and | QRS and T-wave |
| | RR intervals for each | morphology must be |
| | beat are plotted | accurate to determine |
| | against each other and | an accurate QT/RR |
| | the slope is calculated. | ratio. The morphology |
| | Used as an indication | should be sufficiently |
| | of QT adaptation based | represented if the |
| | on rate or circadian | requirements of T-wave |
| | changes. This has been | alternans are met |
| | shown to be modulated by | (listed above). |
| | sympathetic/ | |
| | parasympathetic | |
| | activation and may | |
| | indicate risk of SCD | |
| | when QT/RR slope is | |
| | prolonged. | |
| QT | QT intervals from | ECG recorded from |
| Dispersion | ECG are measured using | multiple vectors. |
| | consistent fiducial | Ideally would have the |
| | points on the Q-wave | standard twelve leads, |
| | and T-wave. | but need to have at |
| | These are computed | least 3 orthogonal leads. |
| | across multiple ECG | QRS and T-wave morphology |
| | vectors and the | must be accurate to |
| | range of QT intervals | determine Q-T intervals |
| | across all vectors is | accurate. The morphology |
| | computed. | should be sufficiently |
| | An increased range of | represented if the |
| | QT intervals across all | requirements of T-wave |
| | vectors is an | alternans are met |
| | indication of SCD | (listed above). |
| | risk due to marked | |
| | heterogeneity of | |
| | repolarization. | |
| QT and/or | Similar to T-wave | Same as T-wave |
| T-wave | alternans, but this | alternans |
| morphology | method looks for changes | |
| | in QT or T-wave | |
| | morphology that do NOT | |
| | exhibit an alternating | |
| | pattern. Various | |
| | approaches quantify | |
| | changes in morphology | |
| | of the QT and T-wave | |
| | segments of the ECG. | |
| | This metric provides | |
| | similar clinical | |
| | information as T-wave | |
| | alternans. | |
| Heart rate | The R-R intervals from | A short segment of |
| turbulence | an ECG strip in which | ECG (approximately 15-20 |
| | a PVC occurred is | beats) after a PVC and |
| | analyzed. The response | 2 beats prior |
| | of the R-R intervals | Markers and measured |
| | immediately after a | R-R intervals are |
| | PVC is analyzed and two | especially useful for |
| | metrics are calculated: | heart rate turbulence, |
| | turbulence onset (which | as this analyzes the |
| | is the relative change | rate characteristics |
| | of RR intervals immediate | before and after a PVC |
| | before and after a PVC) | |
| | and turbulence slope | |
| | (which is the deceleration | |
| | rate of R-R intervals | |
| | after the initial onset | |
| | change). | |
| | Turbulence onset and | |
| | slope infers baroceptor | |
| | reflex by observing the | |
| | modulation of heart rate | |
| | immediately after the | |
| | compensatory pause that | |
| | follows a PVC. (The | |
| | compensatory pause | |
| | allows for increased | |
| | diastolic filling, | |
| | leading to increased | |
| | stroke volume in the | |
| | systolic contraction | |
| | following the pause. | |
| | This stroke volume | |
| | impulse initiates the | |
| | baroceptor reflex.) The | |
| | baroceptor reflex is | |
| | indicative of risk of | |
| | SCD after an | |
| | ischemic event. | |

In some embodiments, the invention includes an implantable medical device programmed to store a physiological signal in response to at least one of a plurality risk stratification measurement triggers. For example, two, three, four, or more, risk stratification measurement triggers can be provided. These triggers prompt the device to save a signal that is useful in risk stratification techniques. In some embodiments, the IMD saves an ECG signal that is adequate to support six of the eight most common risk stratification approaches as discussed in Table 1 with four risk stratification triggers. For example, the device could be adapted to store signal based on one or more of risk stratification measurement triggers including a resting sinus rhythm trigger, a moderate exercise sinus rhythm trigger, a heavy exercise sinus rhythm trigger, and a premature ventricular contraction (PVC) trigger. These triggers cause the device to record an ECG signal useful for implementing many or all of the risk stratification techniques discussed in Table 1, as well as others.

In some embodiments, the device can include a resting sinus rhythm trigger. With such a trigger, during resting sinus rhythm a ECG signal (e.g., about 3 to 10 minutes long) is stored. “Normal sinus rhythm” can be defined as a rate consistently between two programmable rate cutoffs (for example, 50 bpm to 90 bpm). This trigger will provide an ECG signal suitable for the T-wave alternans, ischemia detection via ST segment analysis, signal-averaged QRS complex, QT Dynamicity, and QT and/or T-wave morphology analysis risk stratification metrics.

In some embodiments, the device can include a moderate exercise sinus rhythm trigger. With such a trigger, during normal sinus rhythm at a moderate exertion level an ECG signal (for example, about two minutes long) is stored. “Moderate exercise sinus rhythm” can be defined as a rate consistently between the fast end of the resting sinus rhythm trigger and a second programmable rate cutoff (for example, 90 bpm to 120 bpm). This trigger will provide an ECG signal suitable for the T-wave alternans, ischemia detection via ST segment analysis, QT wave morphology, and T-wave morphology analysis risk stratification metrics.

In some embodiments, the device can include a heavy exercise sinus rhythm trigger. With such a trigger during normal sinus rhythm at a heavy exertion level an ECG signal (for example, about two minutes long) is stored. “Heavy exercise sinus rhythm” can be defined as a rate consistently between the fast end of the moderate sinus rhythm trigger and the VT arrhythmia rate cutoff (for example, 120 bpm to 180 bpm). This trigger will provide an ECG signal suitable for the T-wave alternans, and ischemia detection via ST segment analysis, and QT and/or T-wave morphology analysis risk stratification metrics.

In some embodiments, the device includes a PVC trigger. In such a device, when a PVC is detected a short ECG strip encompassing 10 seconds prior and 50 seconds after the PVC event is stored. A PVC can be detected using any suitable PVC detection methods. For example, a PVC could be defined as any ventricular event whose R-R interval is a programmable percentage shorter than the current four beat R-R average. This trigger will provide an ECG signal suitable for the heart rate turbulence metric.

The signal these triggers cause the IMD to record can be any physiological signal suitable to implement any or all of the risk stratification techniques discussed in Table 1. For example, the signal can include a ECG signal having a single channel, 0.5-95 Hz bandwidth, 256 Hz sampling rate, 0.815 uV digital resolution, and 1.5 uV root-mean-square noise level. Further, in some embodiments, R-waves are automatically detected which allows the device to provide MarkerChannel™ and a beat-by-beat indication of ventricular heart rate/R-R interval.

Some embodiments of the invention further include a memory prioritization scheme to allow the data most likely to be helpful in risk stratification to be stored for processing. In some embodiments, the scheme includes differences in initial memory allocation for data recorded at the prompt of different triggers. For example, in devices having risk stratification measurement triggers comprising resting sinus rhythm trigger, moderate exercise sinus rhythm trigger, heavy exercise sinus rhythm trigger, and PVC trigger, relatively less memory allocation could be provided for resting sinus rhythm triggers than the others. In some embodiments, less allocation is provided for resting sinus rhythm trigger, and relatively more allocation is provided for moderate exercise sinus rhythm trigger, heavy exercise sinus rhythm trigger, and PVC trigger.

Further, the prioritization scheme can allow for signal stored in response to one trigger to replace signal stored in response to a second trigger. For example, if all allocated memory for each category is full, the device can be programmed to allow signal from one category to replace signal from another category. For example, in devices having resting sinus rhythm triggers, moderate exercise sinus rhythm triggers, heavy exercise sinus rhythm triggers, and PVC triggers, the IMD can be programmed to not write over signal stored in response to the moderate exercise sinus rhythm trigger, heavy exercise sinus rhythm trigger, or PVC trigger with signal stored in response to the resting sinus rhythm trigger. In other embodiments, the IMD can be programmed to write over signal stored in response to the resting sinus rhythm trigger if the allocation for signal stored in response to the moderate exercise sinus rhythm trigger, heavy exercise sinus rhythm trigger, or PVC trigger is full.

The prioritization scheme can also provide an algorithm for storage within each trigger category, and each trigger category can have the same or different algorithms. In such embodiments, ECG signals for a given trigger would be stored until the memory allocated to that trigger is used up; at that point, predetermined priority criteria can determine which signals are stored for later retrieval by a user. Therefore, several priority criteria can be defined to determine which signals are stored in the event of the IMD memory being filled.

Any suitable priority criteria could be utilized. Examples of priority criteria include the following; the most recent instance of a trigger criteria being met, the oldest instance of a trigger criteria being met, and the trigger criteria being met for the maximum amount of time during the stored ECG strip. Other priority criteria include the trigger criteria being met selecting it as the strongest over all instances. For example, with a normal sinus rhythm strip programmed to trigger between 50 bpm and 90 bpm, the instance for which the median or average rate over the strip's duration that was closest to 70 bpm (which is the midpoint between 50 bpm and 90 bpm) would be considered to have met the trigger criteria the strongest. Another example of priority criteria, especially for moderate- and heavy-exercise sinus rhythm strips, include the instance with the highest activity level (as measured by the implanted accelerometer). Other priority criteria, especially for sinus rhythm strips, include the instance with R-R interval variability that is most indicative of normal sinus rhythm (i.e., moderate amount of variability due to autonomic tone). Another example of priority criteria includes the instance with the lowest measured noise level. This could be determined by the instance with the smallest time consumed by noisy intervals or any other suitable noise metric.

In some embodiments, as discussed with reference to FIG. 2, the IMD can be adapted to transfer the stored signal to an external device to undertake the actual risk stratification analysis. In such embodiments, the IMD stores the relevant signal as discussed above and transfers it for the risk stratification processing. This approach saves substantial battery life compared to processing the signal within the IMD. For example, the stored signal could be transferred from the IMD to a Medtronic 2090 or CareLink for post-processing. In such embodiments the risk stratification analysis could be performed at any suitable time.

In other embodiments, the signal is translated to a common data format for software analysis. In such embodiments the data is transferred to software that has pre-existing tools for automatic Holter ECG analysis. An example of a common software platform for this analysis is the Phillips Medical Holter Analysis System. For example, the IMD could store signal in a way that facilitates data transfer and translation, such as translation to XML. XML is an open-source data format that enables data to be easily transferred among software platforms. If the software does not directly read XML, software tools can be used to provide translation from the XML format to the proprietary format favored by the analysis software.

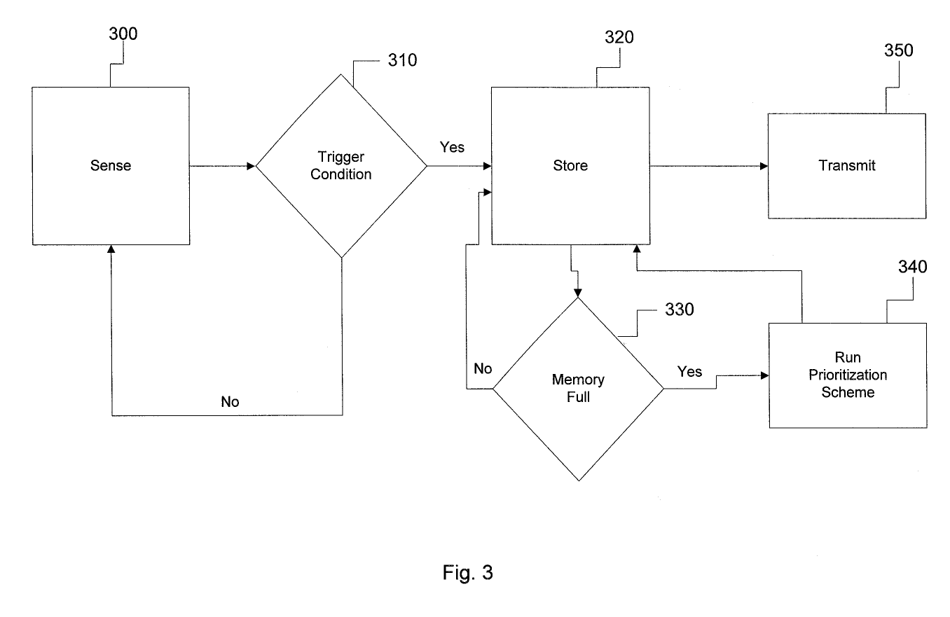

The invention also includes methods of making and implementing any of the various IMDs discussed herein. As shown in FIG. 3, some methods in accordance with embodiments of the invention include the step of sensing a physiological signal (e.g., R-waves), as depicted in block 300. The information sensed can be analyzed by the IMD to determine if a trigger condition is met, as depicted in block 310. The trigger conditions can be any condition adapted to provide useful information for supporting a risk stratification for sudden cardiac death technique, such as those discussed above. If a trigger condition is not met, the IMD continues to sense and does not store the signal. If a trigger condition has been met, the IMD stores the signal, as depicted in block 320.

In some embodiments, the method includes checking the memory of the IMD to determine if it is full, as depicted in block 330. If the memory is not full, the IMD will continue to store the signal. If the memory is full, the IMD can run a memory prioritization scheme such as those discussed above, as depicted in block 340. The device will continue to store the signal in accordance with the parameters of the memory prioritization scheme.

In some embodiments the method includes the step of transmitting the stored signal, as depicted in block 350. The signal could be transferred to any external device as discussed above. In such embodiments the actual risk stratification analysis is performed externally of the IMD. After the signal is transferred and the appropriate risk stratification technique is used, a clinician could help determine whether a patient is at risk for a sudden cardiac death.

Thus, embodiments of the METHOD AND SYSTEM FOR TRIGGERING AN IMPLANTABLE MEDICAL DEVICE FOR RISK STRATIFICATION MEASUREMENTS are disclosed. One skilled in the art will appreciate that the invention can be practiced with embodiments other than those disclosed. The disclosed embodiments are presented for purposes of illustration and not limitation, and the invention is limited only by the claims that follow.

1. An implantable medical device comprising a sensor, a hermetically sealed enclosure, and memory disposed within the hermetically sealed enclosure, wherein the device is programmed to identify the presence of at least one of a plurality of risk stratification measurement triggers and to store a physiological signal sensed by the sensor in response to identification of the at least one risk stratification measurement trigger.

2. The implantable medical device of claim 1, wherein the risk stratification measurement triggers include at least one trigger selected from the group consisting of a resting sinus rhythm trigger, a moderate exercise sinus rhythm trigger, a heavy exercise sinus rhythm trigger, and a PVC trigger.

3. The implantable medical device of claim 2, wherein the physiological signal stored in response to the resting sinus rhythm trigger includes an electrocardiogram signal suitable for a risk stratification measurement selected from the group consisting of T-wave alternans, ischemia detection via ST segment analysis, signal-averaged QRS complex, QT dynamicity, QT wave morphology analysis, and T-wave morphology analysis.

4. The implantable medical device of claim 2, wherein the physiological signal stored in response to the moderate exercise sinus rhythm trigger includes an electrocardiogram signal suitable for a risk stratification measurement selected from the group consisting of T-wave alternans, ischemia detection via ST segment analysis, QT wave morphology analysis, and T-wave morphology analysis.

5. The implantable medical device of claim 2, wherein the physiological signal stored in response to the heavy exercise sinus rhythm trigger includes an electrocardiogram signal suitable for a risk stratification measurement selected from the group consisting of T-wave alternans, ischemia detection via ST segment analysis, QT wave morphology analysis, and T-wave morphology analysis.

6. The implantable medical device of claim 2, wherein the physiological signal stored in response to the PVC trigger includes an electrocardiogram signal suitable for a heart rate turbulence metric risk stratification measurement.

7. The implantable medical device of claim 1, further including a prioritization scheme.

8. The implantable medical device of claim 7, wherein the prioritization scheme includes differences in initial memory allocation.

9. The implantable medical device of claim 7, wherein the prioritization scheme allows signal stored in response to a first trigger to replace signal stored in response to a second trigger.

10. The implantable medical device of claim 7, wherein the prioritization scheme provides an algorithm for storage.

11. The implantable medical device of claim 10, wherein the prioritization scheme provides different algorithms for different triggers.

12. The implantable medical device of claim 1, wherein the physiological signal is an electrocardiogram.

13. An implantable medical device comprising at least two electrodes, a hermetically sealed enclosure, and memory disposed within the hermetically sealed enclosure, the at least two electrodes adapted to sense an electrocardiogram signal, the device programmed to identify the presence of at least one of four risk stratification measurement triggers, the device programmed to store the electrocardiogram signal in response to the identification of one of the at least four risk stratification measurement triggers.

14. The implantable medical device of claim 13, wherein the risk stratification measurement triggers comprise a resting sinus rhythm trigger, a moderate exercise sinus rhythm trigger, a heavy exercise sinus rhythm trigger, and a PVC trigger.

15. The implantable medical device of claim 14, further comprising a prioritization scheme, wherein the prioritization scheme provides less allocation for storing signal in response to the resting sinus rhythm trigger than the moderate exercise sinus rhythm trigger, heavy exercise sinus rhythm trigger, and PVC trigger.

16. The implantable medical device of claim 14, further comprising a prioritization scheme, wherein the prioritization scheme is programmed to not write over signal stored in response to the moderate exercise sinus rhythm trigger, heavy exercise sinus rhythm trigger, or PVC trigger with signal stored in response to the resting sinus rhythm trigger.

17. The implantable medical device of claim 14, further comprising a prioritization scheme, wherein the prioritization scheme is programmed to write over signal stored in response to the resting sinus rhythm trigger if allocation for the moderate exercise sinus rhythm trigger, heavy exercise sinus rhythm trigger, or PVC trigger are full.

18. The implantable medical device of claim 13, further comprising a prioritization scheme, wherein the prioritization scheme includes one or more prioritization criteria selected from the group consisting of a most recent instance of a trigger criteria being met, an oldest instance of a trigger criteria being met, a trigger criteria being met for the maximum amount of time during the electrocardiogram signal, a trigger criteria being met the strongest over all instances, an instance with the highest activity level, an instance with R-R interval variability being most indicative of normal sinus rhythm, an instance with the lowest measured noise level, and combinations thereof.

19. A method of storing information to support risk stratification measurements, the method comprising the steps of sensing a physiological signal, identifying the existence of at least one of a plurality of risk stratification measurement triggers, and storing the physiological signal in response to the identification of the at least one of the plurality of risk stratification measurement triggers.

20. The method of claim 19, further comprising the step of transferring the signal for processing.
